SEQUENCE LISTING (6 sequences)

SEQ ID NO: 1 - DNA, 37 bp, Salmonella typhimurium
cccccgtgcagaattcgaggtcgacggatccgggggg

SEQ ID NO: 2 - DNA, 14 bp, Salmonella typhimurium
  misc_feature (14): The end of sequence No. 2 is joined to the beginning of sequence No. 3 by a single biotin molecule.
cagatgcacgtgag

SEQ ID NO: 3 - DNA, 4 bp, Salmonella typhimurium
tcgc

SEQ ID NO: 4 - DNA, 19 bp, Salmonella typhimurium
gccccgaagaacgttttcc

SEQ ID NO: 5 - DNA, 39 bp, Salmonella typhimurium
  misc_feature (39): The end of Sequence No. 5 carries a biotin molecule.
ctacggtaccgaaacgcgtgtcgggcccgcgaagcttgc

SEQ ID NO: 6 - DNA, 43 bp, Salmonella typhimurium
catggatgccatggctttgcgcacagcccgggcgcttcgaacg

(19) United States
(12) Patent Application Publication
(10) Pub. No.: US 2002/0160485 A1
(43) Pub. Date: Oct. 31, 2002

(54) Polynucleotide motor, a motor system, their preparation and uses

(76) Inventor: Keith Firman, Portsmouth (GB)

Correspondence Address: NIXON & VANDERHYE P.C., 8th Floor, 1100 North Glebe Road, Arlington, VA, 22201-4714, US

(21) Appl. No.: 09/992,028

(22) Filed: Nov. 26, 2001

(30) Foreign Application Priority Data
May 25, 1999 (GB) 9912179.0
May 25, 2000 (GB) PCT/GB00/02034

(51) Int. Cl. C12Q 1/68; C12N 9/22; F02B 1/00; F01K 1/00; F03H 1/00

(52) U.S. Cl. 435/199; 435/6; 60/721

(57) ABSTRACT

A polynucleotide motor is disclosed, comprising an enzyme capable of binding to a nucleic acid sequence, which enzyme is also capable of translocating the nucleic acid sequence without causing cleavage thereof. The motor may be associated with a substance bound to the nucleic acid sequence so that the bound substance, such as a magnetic bead, biotin, streptavidin, a scintillant or the like, can itself be translocated, relative to the region of binding of the enzyme, during translocation. Such a system has applications in the screening or testing for a pre-determined biological, chemical or physical activity; for example, in screening for new pharmacologically-effective ligands.

The present invention relates to a nucleic acid sequence having bound thereto a particular complex involving a subunit of a restriction endonuclease, which complex is capable of translocating the polynucleotide without causing cleavage thereof; and its use, inter alia, in a molecular machine system.

Molecular machines have been described as molecules—on a nanometric scale—that have moving parts and do useful work. A molecular machine system may therefore be a multi-component molecular machine. For such a machine or machine system to operate successfully, it must be based on a compact, stable molecular structure. Accordingly, theoretical studies of molecular machine systems have focused on inflexible, covalent structures, such as graphite- and diamond-like materials, working in a vacuum. However, it is unlikely that such theoretical systems can be built, in practice, in the near future.

On the other hand, the art of preparing polymeric structures is comparatively well-advanced. The drawback of these, however, is that they must fold appropriately in order to provide a usable structure. Protein folds, for example, are difficult to design in view of the lack of strong, natural complementarity of individual amino acids. Contrastingly, work has been carried out which shows that it is possible to design DNA-based structures, so that nucleic acids could be engineered to serve as scaffolds for complex molecular motor—and other—systems. The problem then is to provide a suitable motor or machine system that can appropriately interact with a DNA-based structure.

The study of molecular motors has mainly revolved around muscle proteins and similar macromolecular systems. However, biological motors also exist at the molecular level, and may provide suitable models for the developing nanotechnology industry. Of these, perhaps the most interesting from a biotechnological viewpoint (eg because of the potential use of the information content of DNA at the nanotechnological level) are those enzymes that manipulate nucleic acids. These include RNA polymerases; some enzymes involved in recombination (eg RecBCD); topoisomerases; and type I and III restriction enzymes. However, despite the potential of translocation, the mechanism by which DNA is moved through the protein complex is poorly understood. Furthermore, these enzyme systems are known not only to cause movement or tracking of the DNA, but also to have other effects, such as synthesis (in the case of polymerases); unwinding or breaking of DNA strands (such as by helicases); and cleavage (in the case of the restriction enzymes). Such effects clearly may render these systems undesirable or impossible to use as part of a molecular machine or machine system.

Nevertheless, the present invention surprisingly relates to a motor or machine system that is based on the movement or tracking of an enzyme, particularly a type I restriction enzyme, along DNA.

Type I restriction and modification (R-M) enzyme systems protect the bacterial cell against invasion of foreign DNA (such as viruses) by cleaving DNA which lacks a target specific N6-adenine methylation. The second physiological role of these systems is to restore full methylation of the target sites on the host DNA after DNA replication. Type I R-M enzymes (restriction endonucleases) are distinguished by the fact that the binding of an unmethylated recognition site elicits DNA cleavage at a distantly-located, non-specific site on the DNA. ATP, which is required for DNA restriction, fuels translocation by the enzyme of the DNA from the recognition site to the site of cleavage.

Type I restriction endonucleases specifically recognise a non-palindromic DNA sequence (eg GAAnnnnnnRTCG for EcoR124I, where n is any base and R is a purine). Binding of the endonuclease to a non-modified recognition site activates a powerful ATPase activity, which fuels DNA translocation past the DNA-enzyme complex, while the enzyme remains bound to the recognition site. DNA is cleaved at positions where the DNA translocation stops—either due to a collision of two translocating enzyme molecules on two-site, linear DNA substrates, or due to the build-up of topological strain on circular molecules. The endonuclease does not turn over in the cleavage reaction; however, the ATPase activity continues for a long period of time after the cleavage is completed. DNA methylation activity of the type I R-M systems results in a transfer of a methyl group from a cofactor (S-adenosyl methionine or ‘SAM’) to the N-6 position of a specific adenine in each strand of the recognition sequence. Clearly, the cleavage that is associated with such translocation would be highly likely to negate the usefulness of such an enzyme as a potential motor.

Type I restriction-modification enzymes are composed of three different subunits (HsdR, HsdM and HsdS) encoded by the three hsd genes. All three subunits are absolutely required for restriction activity, while the HsdM and HsdS subunits are sufficient for modification activity and can also form an independent MTase. Type I R-M systems are grouped into four families, based on allelic complementation, protein homologies and biochemical properties of the enzymes. Type IA, IB and ID R-M systems are chromosomally encoded, while most type IC R-M systems are carried on large conjugative plasmids. The type IA family is typified by the EcoKI and EcoBI enzymes, type IB by EcoAI and type IC by EcoR124I. EcoKI forms a stable R₂M₂S₁ complex; however, the independent EcoKI MTase (M₂S₁) is a relatively weak complex, dissociating into an inactive M₁S₁ species and free HsdM subunit. The purified EcoBI restriction endonuclease exists in a number of different stoichiometric forms including R₂M₂S₁, R₁M₂S₁ and R₁M₁S₁. The type IB restriction endonuclease EcoAI is a weak complex that dissociates into MTase and HsdR subunit when purified.

It has already been shown (Janscak in Nucleic Acids Research 26 (19) 4439-4445 (1998), the contents of which are hereby incorporated by reference in their entirety) that the purified EcoR124I restriction endonuclease is a mixture of two species, which have a subunit stoichiometry of R₂M₂S₁ and R₁M₂S₁, respectively. Only the former species was found to have endonuclease activity. However, the R₂M₂S₁ complex is relatively weak, dissociating into free HsdR subunit and the restriction-deficient R₁M₂S₁ assembly intermediate, which appears to be a very tight complex. Although the R₁M₂S₁ complex cannot cleave DNA, it is capable of nicking one strand of the DNA. However, up until the present invention, there had been no indication that the R₁M₂S₁ complex is itself capable of translocating the DNA in spite of the fact that it does not cause cleavage thereof.

For one thing, no satisfactory method had yet been found for producing the restriction-deficient R₁M₂S₁ complex (“the R₁ complex”) preferentially over the R₂M₂S₁ endonuclease, to enable—in practice—synthesis of an R₁M₂S₁ enzyme-polynucleotide complex on a useful scale. We have now found, as described further below in Example 1, that the synthetic Stp-like polypeptide, Stp₂₋₂₆, shifts the equilibrium between the HsdR₂M₂S₁ and HsdR₁M₂S₁ subunit complexes towards the latter form. Stp polypeptide is the anti-restriction determinant of bacteriophage T4 having 26 amino acids, whose presence results primarily in the R₁M₂S₁ restriction-deficient complex.

In addition, we have produced a hybrid HsdR subunit that has the same amino acid sequence as that predicted for the HsdR subunit of EcoprrI. Studies with a hybrid endonuclease comprising the MTase from EcoR124I and the HsdR(prrI) subunit have shown that this hybrid enzyme can only cleave DNA in the presence of extremely high concentrations of HsdR(prrI), which indicates that this assembly has an even weaker R₂-complex than that of EcoR124I and would also be suitable for R₁-complex production. Furthermore, a point mutation of EcoAI has been shown to translocate without cleavage (Janscak et al in Nucleic Acids Research 27(13), 2638-2643) (1999)); single amino acid substitutions in the HsdR subunit of the type IB restriction enzyme EcoAI uncouple the DNA translocation and DNA cleavage activities of the enzyme and could also be a motor of this type.

With the ability preferentially to produce the R₁ complex, the production of a polynucleotide, such as DNA, having complexed therewith an enzyme, such as one comprising the R₁ complex, has been achieved and has surprisingly been found to be capable of translocating the polynucleotide, in spite of the fact that it is not able to cause cleavage thereof.

We have therefore now identified a complex between a polynucleotide sequence, such as a DNA sequence, and an enzyme, such as R₁M₂S₁, capable of translocating the nucleic acid sequence without causing cleavage thereof or other apparent effects that would detract from its usefulness, such as polymerase activity. Furthermore, we have also found that such a (translocation but non-restriction) enzyme-polynucleotide complex can provide the motor for use in the machinery according to the present invention, which motor may be powered, for example, by the presence of ATP and magnesium ions (Mg++).

Accordingly, the present invention provides a polynucleotide motor, comprising an enzyme capable of binding to a nucleic acid sequence, which enzyme is also capable of translocating the nucleic acid sequence without causing cleavage thereof.

Furthermore, the present invention provides an enzyme capable of binding to a nucleic acid sequence, which enzyme is not capable of restriction of the sequence, characterised in that the enzyme is capable of translocating with respect to the sequence. Preferably, the enzyme is also capable of nicking the sequence; that is, in the case of a DNA sequence, of breaking one of the double strands of the DNA sequence without breaking the other strand thereof.

Accordingly, the present invention further provides a polynucleotide motor system comprising a nucleic acid sequence having bound thereto

(a) an enzyme capable of translocating the nucleic acid sequence without causing cleavage thereof; and

(b) a bound substance capable of remaining bound to the nucleic acid sequence during translocation,

whereby, the bound substance is itself translocated, relative to the region of binding of the enzyme to the nucleic acid sequence, during translocation.

Preferably, the nucleic acid sequence is a polynucleotide, such as DNA. The DNA may comprise linear or circular DNA; more preferably, linear DNA.

Preferably, the enzyme is derived from a restriction enzyme, such as one derived from the HsdR subunit, such as one derived from a type I restriction-modification enzyme; more preferably it is derived from a type IC R-M enzyme, such as EcoR124I or EcoprrI. The importance of HsdR as the subunit responsible for DNA cleavage and ATP-binding suggests other approaches that should produce a restriction enzyme, which can translocate DNA without cleavage. One method would be to produce a mutation within the hsdR gene that inactivates the DNA cleavage event without losing the ATPase activity. A restriction enzyme comprising such a mutant HsdR subunit would also be a molecular motor, equivalent in many respects to the R₁-complex; although it will produce bi-directional translocation. A short motif of amino acids, common to many endonucleases, would be the most likely site for such mutations. Other mutations may exist that alter the subunit assembly of the R₂-complex and stabilise the R₁-complex in a manner similar to that observed with HsdR(prr); such mutants would therefore also produce a useful molecular motor. Especially preferred is when the enzyme is derived from a type I endonuclease and exhibits the stoichiometric form R₁M₂S₁, especially the R₁M₂S₁ derived from EcoR124I.

Accordingly, the present invention further provides the use of the R₁-complex for the preparation of a polynucleotide motor in which a nucleic acid sequence (eg a polynucleotide (eg DNA)) to which it is bound or complexed is translocated but not cleaved. Hence, a particularly preferred aspect of the present invention comprises an R₁M₂S₁-DNA complex having bound thereto a substance capable of remaining bound to the DNA during translocation of the DNA, whereby the bound substance is itself translocated during translocation of the DNA.

Hereinafter, the nucleic acid sequence having the (translocating but not restricting) enzyme bound thereto may be referred to as “the polynucleotide-enzyme complex”. In this context, references to a polynucleotide may include references to other nucleic acid sequences, unless specifically stated to the contrary.

Therefore, with the polynucleotide-enzyme complex able to translocate the bound substance, there is further provided a method for translocating a substance bound to a polynucleotide from a distal region of the polynucleotide towards a proximal region, which method comprises

(a)

(i) providing at the distal region of the polynucleotide a bound substance, or

(ii) binding to the distal region of the polynucleotide a substance; and

(b)

(i) providing at the proximal region a complex of the polynucleotide with an enzyme, or

(ii) complexing to the proximal region of the polynucleotide an enzyme, which enzyme is capable of translocating the polynucleotide without causing cleavage thereof; and

(c) activating the enzyme, whereby the enzyme translocates the polynucleotide, including the bound substance, from the distal region towards the proximal region.

Activation of the enzyme will depend upon the particular enzyme chosen and, in the case of the R₁M₂S₁ complex, will conveniently comprise the presence of ATP, as demonstrated in Example 2 below, where the ability of an R₁M₂S₁-DNA complex to translocate, in the presence of ATP, a bound substance comprising a XhoI restriction site linked to a chemiluminescent enzyme is shown. In Example 2, the ATP is present with Mg⁺⁺ in a restriction or cleavage buffer of particular composition. However, the person skilled in the art will understand that a range of buffers or buffer conditions, determinable by routine trial and error, will be suitable for activating the enzyme according to step (c) of the method of this invention. Preferred buffers, though, include freshly-prepared dithiothreitol, and ATP is added at a concentration of preferably greater than 0.5 mM, with about 2 mM being sufficient to result in full enzyme activity.

The bound substance will depend upon the particular use to which the polynucleotide motor system according to the present invention is to be put, which is described further below. The bound substance may itself comprise one or more components or ligands; therefore, the bound substance may comprise:

(a) a substance which is required to be translocated; or

(b) means for binding the substance (which is required to be translocated) to the polynucleotide-enzyme complex; or

(c) both (a) and (b) together.

For example, the bound substance may initially comprise a binding ligand that can bind to a substance in solution, such as a test compound or other material required to become attached to the polynucleotide-enzyme complex (such as chemiluminescent enzymes, magnetic beads or carbon-based ‘gears’) which, once attached, together with the ligand themselves, form the bound substance; or the bound substance may comprise a specific DNA sequence to which DNA-binding protein(s) may bind. Alternatively, the bound substance may not involve a binding ligand, but may be directly bound to the polynucleotide, such as fluorophors or the like. The bound substance therefore may interact with the environment external to the polynucleotide-enzyme complex to produce a detectable and/or measurable effect, such as a chemical, biological or physical reaction, eg the emission of light, electric current or movement.

Many of the uses to which the polynucleotide motor system of the present invention can be put require, in practice, the polynucleotide-translocating enzyme complex to be anchored, such as bound to a solid surface, rather than freely mobile in a solution. Accordingly, the present invention further provides a nucleic acid sequence having bound thereto an enzyme capable of translocating the nucleic acid sequence without causing cleavage thereof (the “polynucleotide-enzyme complex”), which polynucleotide-enzyme complex is attached to a solid support.

Preferably, the nucleic acid sequence also has bound thereto a substance (the ‘bound substance’) capable of remaining bound to the nucleic acid sequence during translocation, whereby the bound substance is itself translocated, relative to the region of binding of the enzyme to the nucleic acid sequence, during translocation. More preferably, the polynucleotide-enzyme complex is substantially linear, prior to translocation; and especially preferred is when only one end thereof is attached to the support, enabling its free end to be available for binding thereto of the bound substance and thus capable of motion relative to the support.

One way of anchoring the polynucleotide-enzyme complex is to bind it to a binding ligand that is itself capable of binding to a substrate coated on the solid support. Hence, the means of attachment between the polynucleotide-enzyme complex and the solid support may be direct or indirect. An example of indirect attachment is wherein the binding ligand is biotin and the substrate is a biotin-binding protein such as avidin, streptavidin or Neutravidin (available from Pierce & Warriner (UK) Ltd, Upper Northgate Street, Chester, UK), any of which may conveniently be coated on a solid support, as described by Bayer et al in A Sensitive Enzyme Assay for Biotin, Avidin, and Streptavidin in Analytical Biochemistry 154 (1), 367-70 (1986). For example, plasmid DNA can be copied (using PCR) to yield a linear DNA fragment, having biotin attached to its end near the recognition site for the EcoR124I enzyme and the DNA can then be attached, via the biotin, to a streptavidin-coated support, followed by assembly of the R₁ complex at the recognition site on the DNA, as described in Example 3.

Conveniently, the solid support comprises a component, such as a chip of a surface plasmon resonance (SPR) machine, which can be used to monitor not only the binding of the polynucleotide-enzyme complex to the support but also reactions of the complex and use of the motor system.

Alternatively, the support or binding ligand may comprise a microparticle containing a scintillant, such as those used in conventional proximity assays.

Accordingly, the present invention further provides a nucleic acid sequence having bound thereto

(a) an enzyme capable of translocating the nucleic acid sequence without causing cleavage thereof;

(b) optionally, a bound substance capable of remaining bound to the nucleic acid sequence during translocation,

whereby, the bound substance is itself translocated, relative to the region of binding of the enzyme to the nucleic acid sequence, during translocation;

(c) a binding ligand, such as biotin, bound to the nucleic acid sequence; and

(d) a substrate, such as avidin or streptavidin, for binding the binding ligand thereto and adapted to anchor the nucleic acid sequence thereto when the nucleic acid sequence is in solution, such as by being coated on the surface of a solid support.

Preferably, the substrate is itself or is associated with solid means for monitoring activity of the polynucleotide-enzyme complex, such as a streptavidin-coated chip of an SPR apparatus.

Depending upon the use to which the polynucleotide motor of the present invention is to be put, the bound substance may itself make use of a binding ligand/substrate (eg biotin/streptavidin or avidin) interaction. For example, the bound substance may itself comprise biotin, or streptavidin or avidin. Commercially-available substances suitable for binding to the polynucleotide-enzyme complex include streptavidin- or biotin-coated magnetic beads, or chemiluminescent enzymes bound to streptavidin or biotin. Clearly, for many uses of the polynucleotide-enzyme complex of the present invention, the biotin and streptavidin or avidin may be interchanged, and the description and examples herein should be interpreted accordingly. Other substances suitable for binding to the polynucleotide-enzyme complex and making use of the biotin-streptavidin or -avidin, or other binding ligand-substrate interactions are available to those skilled in the art, such as DNA-binding proteins or enzymes, which bind specific sequences in the DNA.

A particular example of the use by a polynucleotide motor system according to the present invention of such a bound substance/streptavidin interaction is as a so-called ‘molecular pulley’ or ‘molecular fish-hook and line’. Using the streptavidin-coated SPR chip-immobilised polynucleotide motor system described in Example 2, to the free end of which an oligonucleotide-attached biotin has been ligated, a streptavidin-attached chemiluminescent molecule can be ‘fished’ out of solution by the biotin and, in the presence of ATP to fuel translocation, pulled towards the streptavidin-coated chip, where its presence can be monitored by detection of the emission of light, as described in Example 3. This model of a molecular pulley demonstrates the potential use of the polynucleotide motor-pulley system according to the present invention in pulling molecules, such as a test substance or ligand, out of solution towards a solid surface, where they can be detected and/or isolated and/or their activity measured and/or otherwise tested.

Accordingly, the present invention provides a method for capturing a test substance in solution and bringing it into association with a solid surface, which method comprises

(a) providing a polynucleotide-enzyme complex described above, wherein:

(i) its proximal region is anchored to the solid surface

(ii) its distal region and/or the test substance is/are adapted to enable it to capture the test substance;

(b) bringing the distal region of the polynucleotide-enzyme complex into contact with the test substance, whereby the test substance is captured; and

(c) activating the enzyme, whereby the enzyme translocates the polynucleotide, including the test substance, from the distal region towards the solid surface.

This method has particular application in the pharmaceutical industry, where current screening methods used for testing, eg. protein-drug interactions, rely on two-component systems. In many such systems, interaction between the two components is detected by a mechanism involving light emission produced by a radiolabelled drug or protein interacting with a scintillant, or fluorescence resulting from proximal interaction of the two-component fluorescent chemicals (quantum exchange between these two chemicals results in detectable light emission). Such systems involve, for example, a drug being anchored to the scintillant, which may exist as a bead, or to one of the fluorescent chemicals, and a complex being identified by light emission when a known control protein, which is either radioactive or carries the other fluorescent chemical, binds to the drug. Proteins or other test compounds to be screened for their ability to bind the drug are tested for their ability to displace the control protein bound to the drug/bead complex.

However, these detection systems are difficult to set up and it is difficult to isolate the resulting two-component complexes. Also, although the detection of these complexes appears simple, in practice, this has also been found to be less than reliable. In addition, the use of radioactive samples is a problem for reasons of both safety and disposal.

On the other hand, the molecular motor-based ‘fishing hook’ according to the present invention will not only allow relatively easy detection of interaction between many drugs and many proteins by employing an array method with direct detection of drug-protein interaction using a proximity assay, but will also allow isolation of the resulting complex. Attachment of the drug to the end of the DNA can be achieved through ligation of suitable oligoduplex synthesised with bound drug (or through a biotin-streptavidin-drug complex). Furthermore, this system is designed to work at the molecular level and is therefore capable of detecting single molecules interacting with each other.

This technology is a version of Scintillation Proximity Assays (SPAs). The motor would be attached to a bead (see above) and the proteins to be screened for interaction with drugs would be radiolabelled. Since the DNA translocated by the motor could be produced with a wide variety of attached drugs, this system would allow very large-scale screening of expression libraries against large numbers of drugs. In addition, because there is both a time delay and physical movement of the drug-enzyme complex not only will the system allow direct detection of such interactions, but it would also only detect strong binding complexes (as others are more likely to dissociate before detection). This system should greatly advance the current miniaturisation of SPA and lead to use of such assays on a nanometric scale.

Accordingly, the present invention further provides a method for screening a test substance for a predetermined biological, chemical or physical activity, which method comprises:

(a) providing a solution of the test substance, either (i) itself or (ii) in association with a first interactive substance, capable of providing or inducing a detectable reaction in a second interactive substance;

(b) providing a polynucleotide motor system according to the present invention, which is attached to a solid support and wherein

(i) the bound substance is further capable of binding to a test substance exhibiting the predetermined activity; and

(ii) the bound substance is itself or the solid support comprises the second interactive substance;

(c) activating the polynucleotide motor system, such as by bringing it into contact with/the presence of ATP; and

(d) monitoring the presence or absence of the detectable reaction during or after translocation, such as during or after contact of ATP with the polynucleotide motor system.

The present invention therefore further provides a substance (such as a chemical compound) for use in industry, medicine or agriculture, whenever identified or which is capable of being identified by the screening method of this invention.

According to this screening method, for example in the presence of ATP, the polynucleotide motor system of the present invention will result in translocation of the polynucleotide, together with the bound substance to which may be bound a test substance exhibiting the predetermined activity. Translocation of the bound substance-test substance complex will result in the test substance, optionally together with its associated first interactive substance, being pulled towards the solid support and hence the second interactive substance, resulting in the detectable reaction. Alternatively, the bound substance-test substance complex will itself result in the detectable reaction, which will be detected as this complex approaches the solid support.

Examples of detectable and, preferably also, measurable reactions include chemiluminescence, as mentioned with respect to the model system above; magnetism; electric current; chemical reaction; radioactivity; scintillation; or the like.

Accordingly, further uses for the polynucleotide motor according to this invention include its use as an intelligent switch, such as by the use of a fluorescent marker on DNA, which is activated by quantum exchange following movement of the DNA carrying a first fluorophore towards a second fluorophore. The photon emitted by the quantum exchange(s) can be detected and used to operate other equipment. Hence, activation of the equipment can be governed by the DNA sequence, thereby making the ‘switch’ ‘intelligent’. This has applicability to DNA sequence detection on a nanotechnological scale or for programmed control of instrumentation.

Another use arises from the fact that translocation of DNA with an attached magnetic bead would rotate the bead in a spiral motion around the DNA strand due to the helical nature of DNA. Such motion of a magnet around a conductor would produce an electric current. Since there is evidence that DNA can conduct electricity; such a system could provide a molecular dynamo, and the generated electricity could be tapped to switch transistors.

The present invention will now be illustrated by the following non-limiting examples.

### EXAMPLE 1

Preparation of R₁-complex Molecular Motor

### EXAMPLE 1A

Preparation of Plasmid pCFD30

Plasmid pCFD30 is a recombinant plasmid produced by inserting an oligoduplex of formula

CCGTGCAGAATTCGAGGTCGACGGATCCGG

GGCACGTCTTAAGCTCCAGCTGCCTAGGCC

containing a single recognition site (identified in bold typeface) for EcoR124I (the molecular motor) (Taylor et al., Substrate Recognition and Selectivity in the Type IC DNA Modification Methylase M.EcoR124I in Nucleic Acids Research 21 (21) (1993)) into the unique SmaI site of pTZ19R (Mead et al., Single-Stranded DNA ‘Blue’ T7 Promoter Plasmids: A Versatile Tandem Promoter System for Cloning and Protein Engineering in Protein Engineering 1 67-74 (1986))using standard methods described by Maniatis et al in Molecular Cloning: A Laboratory Manual, Cold Harbor Laboratory, New York (1982). The DNA sequence at the SmaI site (below, identified in italics) of pCFD30 was found to be CCCCCGTGCAGAATTCGAGGTCGACGGATCCGGGGGG, which shows the orientation of the EcoR124I recognition sequence in the plasmid.

### EXAMPLE 1B

Preferential Production of the R₁-complex/Molecular Motor

10 nM pCFD30 plasmid DNA, prepared as described above, was incubated at 37° C. in 25 μl of cleavage buffer (50 mM Tris-HCl pH8.0; 1 mM dithiothreitol (DTT); 10 mM MgCl₂; 50 mM NaCl). To this was added 50 nM MTase(R124) and 40 nM HsdR(R124)—these concentrations ensure primarily R₁-complex formation.

To ensure R₁-complex formation, the synthetic Stp-like polypeptide (described by Penner et al in Phage T4-coded Stp: Double-edged Effector of Coupled DNA- and tRNA-Restriction Systems in Journal of Molecular Biology 249 (5), 857-68 (1995)), Stp₂₋₂₆ was used to promote dissociation of any R₂-complex. Accordingly, the same reactions as above were carried out in the presence of 100 nM Stp₂₋₂₆ polypeptide.

Alternatively, the above procedure can be followed, but using 40 nM HsdR(prrI) in place of HsdR(R124).

### EXAMPLE 1C

Confirmation of Production of the R₁-complex

To confirm production of the R₁-complex and to ensure no R₂-complex is present, a cleavage test is performed. If only R₁-complex is present there should be no cleavage product.

2 mM ATP was added to the product of Example 1B to initiate the reaction, and the samples were incubated for 30 minutes. A cleavage-positive control was also performed in which an excess (250 nM) of HsdR (R124) was added in order to promote formation of R₂-complex. 10 μl samples were run on 1% agarose gels.

The R₂-complex was found to cleave the plasmid DNA, as expected. No cleavage was observed for the R₁-complex, confirming production of R₁-complex. Either mixture can be used for all subsequent motor experiments.

### EXAMPLE 2

Translocation of DNA by an R₁-complex

This example provides the first confirmation of DNA translocation by the R₁-complex: circular plasmid DNA carrying a chemiluminescent enzyme was translocated and the translocation shown by cleavage with XhoI. The model used depends upon the fact that a unique restriction enzyme cleavage site (for XhoI) can be ‘buried’ in the translocation complex preventing cleavage by XhoI. In this example, the cleavage of circular DNA carrying a biotin molecule to which a streptavidin-linked chemiluminescent enzyme can be attached was investigated. The presence of the chemiluminescent enzyme should halt translocation by collision with the translocating. R₁-complex and ‘bury’ the XhoI site within the translocation complex. When the XhoI site is buried in the translocation complex (translocation will be stopped by the presence of the chemiluminscent enzyme), there is no linearisation of the plasmid by XhoI. This event is independent of the direction of translocation.

### EXAMPLE 2A

Preparation of DNA-bound Chemiluminescent Enzyme

Plasmid pCFD30 (as defined in Example 1A) was linearised with XmnI and ligated to an excess of oligoduplex (CAGATGCACGTGAG*TCGC) containing a XhoI site (identified by bold typeface) and a single biotin molecule (obtained from Cruachem Ltd, Glasgow) linked to thymine (*T) to produce pCFD30-biotin. Recombinants were identified by XhoI cleavage. The presence of a single copy of the oligoduplex was produced by XhoI cleavage followed by re-ligation and confirmed by DNA sequence analysis of the resulting recombinants.

An excess of streptavidin-linked chemiluminescent enzyme (streptavidin-bound horseradish peroxidase (S-HRP), available from Pierce & Warriner (UK) Ltd, Upper Northgate Street, Chester, UK) was added to the plasmid, and the complex was purified from surplus enzyme using ethanol precipitation (Maniatis, ibid, Example 1A).

The presence of the DNA-bound enzyme was confirmed by a simple chemiluminescent measurement. 100 ng of the pCFD30-biotin/S-HRP plasmid were spotted onto a nylon membrane soaked in chemiluminescent substrate (SuperSignal (Registered Trademark) Substrate from Pierce & Warriner (UK) Ltd, Upper Northgate Street, Chester, UK). The membrane was incubated at 37° C. to activate the enzyme and the spot visualised using X-ray film. Controls of pCFD30 and pCFD30-biotin were spotted onto the same membrane. A positive light emission was obtained for the pCFD30-biotin/S-HRP plasmid only, indicating a DNA-bound chemiluminescent enzyme.

### EXAMPLE 2B

Confirmation of Translocation by R₁-complex

An R₁-complex was produced and confirmed as not cleaving pCFD30 as described in Example 1. This R₁-complex was incubated with an equimolar concentration of pCFD30-biotin with bound chemiluminescent enzyme prepared as above. ATP was added, using the method described in Example 1. After 15 min at 37° C., the plasmid was subjected to cleavage with 10 units of XhoI. In addition, pCFD30, pCFD30-biotin, pCFD30-biotin/S-HRP and pCFD30-biotin/S-HRP with R₁-complex, but without ATP were also subjected to XhoI cleavage.

All plasmids except the pCFD30-biotin/S-HRP with ATP were cleaved by the XhoI, producing linear DNA (detected following gel electrophoresis). The addition of y-S-ATP (a non-hydrolysable analogue of ATP, incapable of supporting translocation) instead of ATP also produced linear DNA. Hence, the XhoI site was ‘buried’ in the stalled translocation complex produced by the action of ATP, preventing cleavage. Translocation in either direction accomplishes this process.

Therefore, the R₁-complex is capable of ATP-driven translocation and can be used as a molecular motor. This is the first confirmation of translocation (as opposed to ATPase activity) by the R₁-complex.

### EXAMPLE 3

Surface-Attached Molecular Motor

Plasmid PCFD30 DNA was copied using PCR (using Universal primer with biotin attached at the 5′-end (available from Cruachem Ltd., Todd Campus, West of Scotland science Park, Acre Road, Glasgow G20 OUA) and a primer overlapping the unique XmnI site: GCCCCGAAGAACGTTTTCC) to yield a linear DNA fragment with biotin attached at one specific end (near the recognition site for the R₁ enzyme). The PCR product was attached to the streptavidin-coated chip of an SPR (surface plasmon resonance) machine (Biacore X available from Biacore AB, Meadway Technology Park, Stevenage, Herts., UK). Attachment was monitored using SPR to confirm that no more PCR product could bind to the chip. Biotin was attached to another oligoduplex (as in Example 2A), which was ligated to the other end of the chip-bound PCR product; again, attachment was monitored using the SPR.

This additional oligoduplex had a restriction enzyme target site near one end, accessible for cleavage by the restriction enzyme (XhoI).

The R₁ complex, prepared according to Example 1 (the molecular motor), was attached to the PCR product (the target site was near the chip) and attachment monitored by SPR. Addition of ATP resulted in translocation of the DNA. Following translocation, the XhoI target site is inaccessible because it is buried in the translocation complex, as seen in Example 2.

To monitor the process of translocation and to confirm the ‘fishing hook’ model, a streptavidin-bound enzyme capable of chemiluminescence (S-HRP, as described in Example 2), was ‘fished’ out of solution by the biotin bound at the end of the DNA molecule. The presence of the enzyme was monitored by light emission from the chip. Cleavage by XhoI releases back to solution all non-translocated chemiluminescent enzyme. Chemiluminescent enzyme present in solution was removed by repeated washing.

### EXAMPLE 4

Further Experiments on Surface Attachment of the Motor

The experiments described below were performed using a streptavidin-coated chip (Biacore SA5 available from Biacore AB, Meadway Technology Park, Stevenage, Herts., UK) from a surface plasmon resonance machine (BIAlite 2000), as this machine allows the real-time monitoring of attachment of molecules to the surface of the chip. It also allows the real-time monitoring of release of molecules from the chip surface.

To further investigate the function of the motor when attached to a solid surface via one end of a DNA molecule, the following experiments were carried out:

### EXAMPLE 4A

Preliminary Surface-Attachment of DNA

An oligonucleotide (CTACGGTACCGAAACGCGTGTCGGGCCCGCGAAGCTTGC_{X}) carrying a biotin molecule (ₓ) at one end was synthesised by Cruachem. Attachment of the oligo to the surface was monitored by SPR. This oligo was annealed to a complementary oligo (CATGGATGCCATGGCTTTGCGCACAGCCCGGGCGCTTCGAACG) to give an oligoduplex with a biotin attached and with a suitable 6-base-pair “sticky-end” at the 3′end (biotin end) to allow ligation of another DNA molecule. Annealing of this second oligo was also monitored by SPR. The running buffer (buffer 1) used was 10 mM Tris-HCl (pH8), 10 mM MgCl₂, 100 mM NaCl, 1 mM DTT

### EXAMPLE 4B

Determination of Optimum Motor Density

Several experiments were performed to determine the maximum density at which such an oligoduplex could be bound to a streptavidin-coated chip of a surface plasmon resonance machine. This was accomplished by repeated passage of dilute (<1 nM, but known concentration) solutions of oligonucleotide over the chip. When no further binding was observed this total concentration was the capacity of the chip. It was confirmed that, at low densities, the remaining streptavidin sites on the chip could be blocked using free biotin and that no more oligoduplex could then be bound to the chip.

However, there was found to be considerable variation in the capacity of each batch of chips presumably due to small variations in the surface area. Therefore, the number of molecules present on each chip tested was calculated. Using these data, it was possible to determine which chips carried approximately one molecule every about 100 nm² on the chip. This was found to be the best density for the experiment described below; at lower densities, any changes in the SPR data were difficult to measure, whereas higher densities gave more random results suggesting interference between adjacent motors/DNA molecules.

### EXAMPLE 4C

Experiments Using Linear DNA

Plasmid pCFD30 DNA was copied using PCR (using universal primer available from Cruachem Ltd., Todd Campus, West of Scotland Science Park, Acre Rod, Glasgow G20 OUA) and a primer overlapping the unique XmnI site: GCCCCGAAGAACGTTTTCC) to yield a linear DNA fragment. This linear DNA was cleaved with KpnI to produce a suitable complementary “sticky-end”. The PCR product was attached to the streptavidin-coated chip of an SPR by ligation to the oligoduplexes attached to the surf ace of the chip using standard procedures described in Maniatis (ibid, Example 1). The chip was washed free of ligase and any unligated linear DNA was removed using 1% SDS in the buffer detailed above (this can also be used to remove EcoR124I enzyme from the DNA on the chip). The data from the SPR showed ligation was successful.

Purified EcoR124I in the form of the R₁-complex (100 nM MTase+100 nM HsdR (R124 with and without 100 nM Stp, or prr[which does not require Stp]), or the R₂-complex (100 nM MTase+500 nM HsdR) in cleavage buffer (Example 1B), was allowed to bind to the DNA (monitored by SPR). Upon addition of 2 mM ATP a large release of material from the chip was observed for both complexes. Confirmation that the motor was presented as an R₁-complex was determined by the method described in Example 1B.

Interestingly, over any time period, there was a greater release of material for the R₂-complex than the R₁-complex and the rate of release was also greater for the R₂-complex. Washing of the chip with buffer 1 (Example 4A), after the translocation assays, followed by further addition of ATP showed that the R₁-complex was capable of further changes but the R₂-complex was not.

This is due to translocation, by the motor, of the DNA followed by displacement of the biotin-streptavidin linkage to the surface, thereby releasing the motor. The R₂-complex is capable of bi-directional translocation and can displace all bound motors, while the R₁-complex is uni-directional and only some (˜50%) of the motors are displaced from the surface. Addition of further ATP to the R₁-complex allows further translocation and thereby further displacement of the motor. Therefore, the R₁-complex is also capable of re-setting after translocation.

### EXAMPLE 4

Conclusion

The translocation of DNA by the motor leads to displacement of the DNA from the surf ace of the chip. Therefore, both R₁-complex and R₂-complex are capable of translocation. The data from the R₁-complex also shows that not all the motors are displaced suggesting that some motors are translocating away from the surface without displacement.

### EXAMPLE 5

Investigation of the Disruption by Translocation of the Biotin-Streptavidin Linkage

Example 2 hereinabove confirmed translocation by the R₁-complex. However, although the concept of the biotin-streptavidin-HRP complex ‘blocking’ translocation appears to be the most logical explanation of these results, another explanation could be that translocation is simply blocked by the lack of available DNA for translocation and by chance the XhoI site becomes ‘buried’ in the resulting stalled complex. Therefore, it was necessary to investigate any displacement of the biotin-streptavidin linkage.

The following experiment was undertaken to show that displacement of the motor (R₁-) from the surface of the SPR chip was not just the result of the collision of the motor with the surface but a result of a displacement of the biotin-streptavidin bond by translocation.

### EXAMPLE 5A

Use of Linear DNA

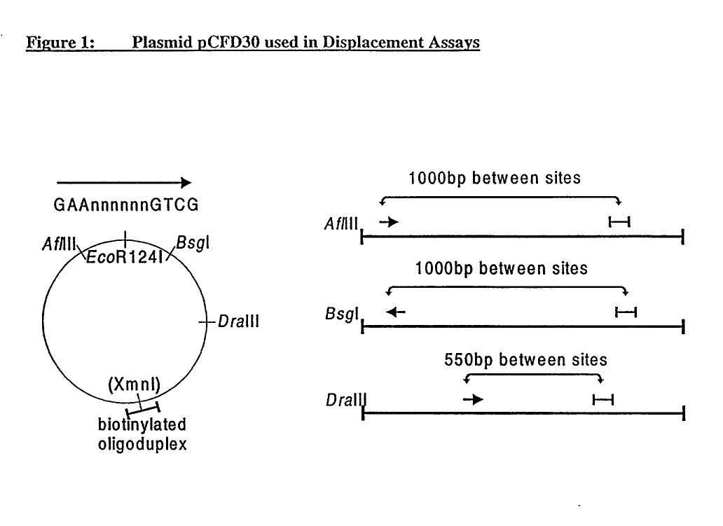

The pCFD30-biotin plasmid, produced as described above, was used to produce three linear plasmids by cleavage with AflIII, BsgI or DraIII, respectively, (shown in FIG. 1). Cleavage of 200 ng of pCFD30-biotin with the respective restriction enzymes (following manufacturer's instructions—New England BioLabs) produced the required plasmids, each having the biotin molecule at different distances (and orientations) from the S_{R124} site. The oligoduplex contains a unique XhoI site, which should be inaccessible if ‘buried’ under the translocating motor/biotin-streptavidin-HRP (as previously described). However, if the biotin-streptavidin bond is broken by the translocating motor, then addition of excess streptavidin, after addition of ATP, should prevent HRP from rebinding to the biotin on the DNA.

Streptavidin-HRP (Pierce & Warriner Cat No. 21126) was added to the plasmid (excess over the plasmid concentration) and excess removed by ethanol precipitation of the DNA using standard techniques (Maniatis ibid, Example 1). The presence of the linked HRP was assayed on a sample (5-10 ng) of DNA using the Pierce Chemiluminescence substrate Supersignal (Cat no. 34080) as described by the manufacturer following “spotting” of diluted samples onto nylon membrane.

The HRP-linked plasmid (100 nM) was incubated with equimolar concentration of MTase+HsdR (R124 or prr) (R₁-complex) motor, as described in Example 2B. Translocation was commenced by addition of 2 mM ATP.

For the blockage of translocation assay (as described in Example 2 for circular DNA), cleavage of the plasmid was assayed using XhoI enzyme followed by agarose gel electrophoresis (Maniatis ibid, Example 1).

For the displacement assay 300 nM streptavidin was added to quench any displaced S-HRP and the presence of S-HRP on the DNA was monitored by ethanol precipitation of the DNA followed by spotting onto nylon membrane and assaying for chemiluminescence as above.

All XhoI digestions produced cleavage of the linear DNA into fragments of the predicted sizes, indicating that translocation did not ‘bury’ the XhoI site. This suggests that the previous results were a consequence of using circular plasmid DNA.

The displacement assays showed a lack of, or significantly reduced amounts of, chemiluminescence indicating loss of HRP from the DNA. Controls of no ATP added, no EcoR124I (motor) and use of y-S ATP (a non-hydrolysable analogue of ATP that prevents translocation) indicated that the excess streptavidin did not displace the bound streptavidin-HRP except when translocation occurred.

### EXAMPLE 5B

Lack of Displacement of Biotin-Streptavidin when Bound Towards End of Linear DNA

The above experiments were repeated but with the biotin attached to the very end of a linear DNA. A modified version of the oligoduplex described in Example 4A was employed for these experiments. The first few bases of each upper strand were altered to produce a PstI “sticky end”.

The experiments were as described in Example 2 except that the oligoduplex was ligated to the PstI site of pCFD30 DNA and, to ensure the correct arrangement of the S_{R124} recognition site and biotin, the plasmid was further cleaved with SalI. Purification of the large fragment using standard techniques of phenol extraction of low melting point agarose gels resulted in a linear DNA molecule with biotin attached to one end and the S_{R124} site at the other end. In this case no displacement of the HRP was observed under any of the conditions described above. Therefore, when the biotin is “free” at the very end of a DNA molecule it is not displaced by the translocating complex.

### EXAMPLE 5

Conclusions

The observation in Example 2 that the XhoI site, present on the circular plasmid DNA, was ‘buried’ by the stalled translocating complex was not due to the presence of the biotin-S-HRP blocking translocation, but to the stalling of the complex following translocation of all available DNA. Nevertheless, Example 2 still demonstrates that translocation, by the R₁-complex, occurs.

Therefore, while we have confirmed that the R₁-complex is capable of translocation, unexpectedly it is also able to displace the very strong biotin-streptavidin linkage. However, when the biotin is attached to any of the terminal few nucleotides of the DNA, the biotin-streptavidin linkage is not disrupted.

The EcoR124 R₁-complex used was produced using excess Stp to ensure little or no R₂-complex was formed. However, the same results were obtained when, in place of HsdR(R124), HsdR(prr) in the absence of Stp was used. Example 3 shows that the R₂-complex is bi-directional; all motors were displaced from the chip surface. It has also been found (Firman et al in Eur Mol Biol Org J 19(9) 2094-2102 (2000)) that the R₁-complex is less processive (resulting in a slower translocation rate) than the R₂-complex.

### EXAMPLE 6

Molecular Dynamo

The plasmid used in example 2A was used to attach a streptavidin-coated paramagnetic bead (available from Pierce & Warriner). DNA carrying the attached bead was purified from solution using a magnet following the manufacturer's instructions. This DNA, linearised by cleavage with PstI and with the attached paramagnetic bead was used to determine whether the molecular motor would rotate the paramagnetic bead in solution.

The DNA and motor were prepared as described in Example 1 and the sample loaded into the capillary tube of a paramagnetic resonance machine. 2 mM ATP was added to one of the tubes and allowed to diffuse into the sample. As the ATP entered the measuring cell, a paramagnetic moment was measured, which gradually weakened as the ATP diffused through the sample. Addition of X-S-ATP produced no such signal, indicating that ATP hydrolysis is required for this effect.

The bead “spinning” within the applied magnetic field produces a paramagnetic moment. This reflects translocation of the DNA (the free end of the DNA rotates as the motor follows the double helix) and could be used to measure translocation. Furthermore, it indicates the possibility of replacing the magnetic bead with a permanent magnetic bead and using this system as a molecule dynamo—if the DNA is surface-attached the spinning magnetic bead should generate electricity within the DNA “conductor”.

It will be apparent to a person skilled in the art that the above-described system has applications in the screening or testing for a pre-determined biological, chemical or physical activity; for example, in screening for new pharmacologically-effective ligands.

1. A type I restriction-modification enzyme capable of binding to a nucleic acid sequence and also capable of translocating the nucleic acid sequence without causing cleavage thereof, said enzyme having HsdR, HsdM and HsdS sub-units, wherein the HsdR sub-unit is of the type IC restriction-modification enzyme EcoprrI or a mutant of said HsdR sub-unit which imparts to the enzyme the property of translocating a nucleic acid sequence without causing cleavage thereof, and without loss of ATPase activity.

2. A molecular motor system comprising a nucleic acid sequence having bound thereto:
(1) at a first, proximal, region of the nucleic acid, an enzyme capable of translocating the nucleic acid sequence without causing cleavage of the nucleic acid during the translocation, said enzyme remaining bound to the nucleic acid, as a complex therewith, at said proximal region, during the translocation; and
(2) at a second, distal, region of the nucleic acid, a bound substance capable of remaining bound to the nucleic acid sequence during the translocation, whereby the bound substance becomes translocated, relative to the region of binding of the enzyme to the nucleic acid sequence, as a result of the translocation of the nucleic acid to which it is bound.

3. A system according to claim 2, wherein the nucleic acid sequence comprises a circular or linear DNA sequence.

4. A system according to claim 2, wherein the enzyme comprises a type I restriction-modification enzyme having HsdR, HsdS and HsdM sub-units.

5. A system according to claim 4, wherein the enzyme comprises a type IC restriction-modification enzyme and exhibits the stoichiometric form HsdR₁M₂S₁.

6. A system according to claim 4, wherein the HsdR sub-unit is that of the type IC restriction-modification enzyme EcoprrI or a mutant of said HsdR sub-unit which imparts to the enzyme the property of translocating a nucleic acid sequence without causing cleavage thereof, and without loss of ATPase activity.

7. A system according to claim 2, wherein the bound substance comprises a binding ligand that can bind to a substance in solution.

8. A system according to claim 2, wherein the nucleic acid is attached to a solid support.

9. A system according to claim 7, wherein the nucleic acid is linear, thus having two ends, the bound substance being bound at one end and a solid support being bound at the other end.

10. A system according to claim 7, wherein the bound substance comprises one or more of:
(a) a substance which is required to be translocated; or
(b) means for binding to the nucleic acid-enzyme complex the substance which is required to be translocated; or
(c) both (a) and (b) together.

11. A system according to claim 7, wherein the bound substance comprises one or more of:
(a) a binding ligand for binding a substance in solution, suspension or dispersion;
(b) an enzyme which produces chemiluminescence;
(c) a magnetic substance;
(d) a DNA sequence;
(e) a scintillant;
(f) a radioactive substance;
(g) a substance capable of producing an electric current;
(h) a substance capable of movement or resulting in movement;
(i) a substance capable of interacting with the environment of the system to produce a detectable and/or measurable effect; and/or
(j) biotin, streptavidin or avidin.

12. A molecular motor system comprising a nucleic acid sequence having bound thereto:
(1) an enzyme capable of translocating the nucleic acid sequence without causing cleavage of the nucleic acid during the translocation, said enzyme remaining bound to the nucleic acid, as a complex therewith, at said proximal region, during the translocation; and
(2) a solid support.

13. A molecular motor system according to claim 12, wherein the enzyme comprises a type I restriction-modification enzyme having HsdR, HsdS and HsdM sub-units.

14. A molecular motor system according to claim 13, wherein the enzyme comprises a type IC restriction-modification enzyme and exhibits the stoichiometric form HsdR₁M₂S₁.

15. A molecular motor system according to claim 13, wherein the HsdR sub-unit is that of the type IC restriction-modification enzyme EcoprrI or a mutant of said HsdR sub-unit which imparts to the enzyme the property of translocating a nucleic acid sequence without causing cleavage thereof, and without loss of ATPase activity.

16. A method for translocating a substance bound to a nucleic acid sequence from a distal region of the nucleic acid sequence towards a proximal region, which method comprises:
(a)
(i) providing at the distal region of the nucleic acid sequence a bound substance, or
(ii) binding a substance to the distal region of the nucleic acid sequence; and
(b)
(i) providing at the proximal region a complex of the nucleic acid sequence with an enzyme, or
(ii) complexing an enzyme to the proximal region of the nucleic acid sequence, which enzyme is capable of translocating the nucleic acid sequence without causing cleavage thereof; and
(c) activating the enzyme, whereby the enzyme translocates the nucleic acid sequence, including the bound substance, from the distal region towards the proximal region.

17. A method according to claim 16, wherein step (c) is carried out in the presence of ATP and Mg⁺⁺ ions.

18. A method according to claim 16, wherein step (c) is carried out in the presence of a restriction buffer.

19. A method according to claim 16, wherein step (c) is carried out in the presence of dithiothreitol.

20. A method according to claim 16, wherein the polynucleotide-enzyme complex is attached to a solid support.

21. A method according to claim 16, wherein the nucleic acid sequence comprises a circular or linear DNA sequence.

22. A method according to claim 16, wherein the enzyme comprises a type I restriction-modification enzyme having HsdR, HsdS and HsdM sub-units.

23. A method according to claim 21, wherein the enzyme comprises a type IC restriction-modification enzyme and exhibits the stoichiometric form HsdR₁M₂S₁.

24. A method according to claim 21, wherein the HsdR sub-unit is that of the type IC restriction-modification enzyme EcoprrI or a mutant of said HsdR sub-unit which imparts to the enzyme the property of translocating a nucleic acid sequence without causing cleavage thereof, and without loss of ATPase activity.

25. A method for capturing a test substance in solution, suspension or dispersion and bringing it into association with a solid surface, which method comprises:
(a) providing a molecular motor system comprising a nucleic acid having:
(i) a proximal region of the nucleic acid to which is bound an enzyme capable of translocating the nucleic acid sequence without causing cleavage of the nucleic acid during the translocation, said enzyme remaining bound to the nucleic acid, as a complex therewith, at said proximal region, during the translocation;
(ii) a distal region of the nucleic acid which enables the test substance to be captured, the distal region and/or the test substance being adapted for that purpose; and
(iii) a solid surface attached to the nucleic acid
(b) bringing the distal region of the polynucleotide-enzyme complex into contact with the test substance, whereby the test substance is captured; and
(c) activating the enzyme, whereby the enzyme translocates the polynucleotide, including the test substance, from the distal region towards the solid surface.

26. A method for screening a test substance for a predetermined biological, chemical or physical activity, which method comprises:
(a) providing a solution, suspension or dispersion of the test substance, either (i) itself or (ii) in association with a first interactive substance, capable of providing or inducing a detectable reaction in a second interactive substance;
(b) providing a polynucleotide motor system comprising a nucleic acid having bound thereto:
(1) at a first, proximal region of the nucleic acid, an enzyme capable of translocating the nucleic acid sequence without causing cleavage of the nucleic acid during the translocation, said enzyme remaining bound to the nucleic acid, as a complex therewith, at said proximal region, during the translocation; and
(2) at a second, distal region of the nucleic acid, a bound substance capable of remaining bound to the nucleic acid sequence during the translocation, whereby the bound substance becomes translocated, relative to the region of binding of the enzyme to the nucleic acid sequence, as a result of the translocation of the nucleic acid to which it is bound,
wherein the nucleic acid is attached to a solid support and wherein
(i) the bound substance is further capable of binding to a test substance exhibiting the predetermined activity; and
(ii) the bound substance is itself or the solid support comprises the second interactive substance;
(c) activating the polynucleotide motor system to effect translocation; and
(d) monitoring the presence or absence of the detectable reaction during or after translocation.

27. An enzyme according to claim 1 bound to a nucleic acid sequence.

28. An enzyme according to claim 27 wherein the nucleic acid sequence comprises a circular or linear DNA sequence.
